(19) United States
(12) Patent Application Publication
(10) Pub. No.: US 2008/0152757 A1
(43) Pub. Date: Jun. 26, 2008

(54) Method of Making Fresh Cheese with Enhanced Microbiological Safety

(76) Inventors: Zuoxing Zheng, Palatine, IL (US); Charles Kennett, Brookfield, IL (US)

Correspondence Address: FITCH EVEN TABIN & FLANNERY, 120 S. LASALLE STREET, SUITE 1600, CHICAGO, IL, 60603-3406, US

(21) Appl. No.: 11/615,428

(22) Filed: Dec. 22, 2006

(51) Int. Cl. A23L 3/34; A01J 25/00; C12P 1/04

(52) U.S. Cl. 426/9; 426/532; 435/170

(57) ABSTRACT

Described herein is a high moisture, high pH fresh cheese having enhanced microbial food safety and methods of making the high moisture, high pH fresh cheese. Enhanced microbial food safety is achieved by using the combination of a low acid, high antimicrobial producing starter culture with food grade antimicrobial agents.

### FIELD OF THE INVENTION

Described herein is a high moisture, high pH fresh cheese having enhanced microbial food safety and methods of making the high moisture, high pH fresh cheese. Enhanced microbial food safety is achieved by using the combination of a low acid, high antimicrobial producing starter culture with food grade antimicrobial agents.

### BACKGROUND OF THE INVENTION

Food processors have long felt the need for methods to prolong the shelf life of foodstuffs. By increasing the amount of time a foodstuff is stable, processors can mitigate inventory losses due to spoiled foodstuffs. Prior methods, such as, the use of packaging, preservatives, and/or specific storage parameters (e.g., refrigeration) have been used to stave off spoilage.

Bacterial contamination of foods is known to be responsible for spoilage and for the transmission of food borne illness. In particular, Listeria monocytogenes has been associated with such foods as raw milk, cheeses (particularly soft-ripened varieties), ice cream, raw vegetables, fermented raw meat sausages, raw and cooked poultry, raw meats (of all types), and raw and smoked fish. Listeria monocytogenes is a psychrotrophic food borne pathogen which is very widespread in the environment and in foods. The ability of Listeria monocytogenes to grow at temperatures as low as 3° C. permits multiplication in refrigerated foods.

Bacteriocins are antimicrobial peptides that are produced by bacteria and which have bactericidal action against closely related species. The most extensively characterized bacteriocin is nisin which is produced by a lactic acid type bacteria and which may be used to prevent growth of Gram-positive bacteria in a wide variety of different food products, particularly dairy products (see, e.g., U.S. Pat. No. 2,744,827; U.S. Pat. No. 4,584,199; U.S. Pat. No. 4,597,972; and U.S. Pat. No. 5,527,505).

Currently, most commercial refrigerated fresh cheese products have a very limited shelf life and do not have secondary barriers to psychrotrophic pathogens. However, there have been efforts to enhance the microbiological safety of fresh cheese products. Generally, known methods to enhance microbiological safety in food products include cooking, adjusting water activity, adjusting pH, or using preservatives. However, these methods are generally expensive and therefore increase the cost of storing and producing the foodstuff while diminishing the organoleptic properties of the food product.

### SUMMARY OF THE INVENTION

Described herein is a fresh cheese product comprising milk ripened with a low acid, high antimicrobial producing starter culture, potassium sorbate, and nisin ingredient, and a pH in the range of about 5.8 and about 6.8, wherein the fresh cheese is stable for at least 30 days and exhibits organoleptic properties similar to traditional fresh cheeses.

In one embodiment, the starting culture is Lactococcus lactis. In another embodiment, the starting bacterial culture is assigned ATCC ______.

In one embodiment, potassium sorbate is added in the range of about 0.01 to about 0.1 percent. In another embodiment, nisin ingredient (generally containing about 2.5 percent pure nisin) is added in the range of about 0.01 to about 0.1 percent. In yet another embodiment, the nisin is Nisaplin®.

Also described herein is a method of making a fresh cheese product comprising adding to milk a low acid, high antimicrobial producing starter culture and adding to the ripened milk potassium sorbate and nisin, wherein the fresh cheese has a pH in the range of about 5.8 and about 6.8 and is stable for at least 30 days and exhibits organoleptic properties similar to traditional fresh cheeses.

Also described herein is a biologically pure culture of Lactococcus lactis having all of the characteristics of strain NB-1, ATCC ______.

Also described herein is a biologically pure culture of low acid, high antimicrobial producing bacteria. In one embodiment, the biologically pure culture of low acid, high antimicrobial producing bacteria is Lactococcus lactis.

### BRIEF DESCRIPTION OF THE DRAWINGS

FIG. 1 is a graph depicting antimicrobial activity over a period of time for a low acid, high nisin producing culture and a conventional culture according to one embodiment of the invention.

FIG. 2 is a graph depicting acid production for three strains of Lactococcus lactis over time.

### DETAILED DESCRIPTION OF THE INVENTION

Described herein is a high moisture, high pH fresh cheese having enhanced microbial food safety and methods of making the high moisture, high pH fresh cheese. Enhanced microbial food safety is achieved by using the combination of a low acid, high antimicrobial producing starter culture with food grade antimicrobial agents.

The invention described herein may be used in the production of any high moisture, high pH, cultured dairy product, such as, but not limited to, fresh Mexican cheeses (such as Queso Fresco), mozzarella cheese, and cottage cheese. The inventive cheeses produced by the inventive methods described herein advantageously extend the shelf life of the cheese and render the cheeses more microbiologically stable. In another embodiment, the inventive processes described herein may be applied to fermented meats, vegetables, and foodstuffs.

Queso Fresco is a creamy, soft, and mild unaged fresh cheese that originated in Mexico. It is made by pressing the whey from rennet coagulated cheese curd. Queso Fresco generally has a high water activity of over 0.89 and a high pH of over 6.0. A typical Queso Fresco cheese has a good melt restriction. That is, when the cheese is exposed to moderate heat treatment such as boiling or microwave for a short period of time, it should remain in good shape and not melted unlike most other cheese products. Because Queso Fresco, like other fresh cheeses, is not cooked or pasteurized, it will spoil in a matter of days without the addition of preservatives. Specifically, Queso Fresco is susceptible to the growth of bacterial pathogens such as Gram-positive bacteria, including Listeria monocytogenes and Clostridium botulinum as well as Gram-negative bacteria such as Escherichia coli O157:H7 and Salmonella. In order to be considered microbiologically stable, the product should not allow the inoculated pathogenic bacteria to grow more than 1 log in cfu/g in at least one month under refrigerated conditions. Most commercial Queso Fresco products on the market today do not meet these criteria.

Generally, the invention is directed to a dairy product fermented with a bacterial culture, which produces a high level of nisin-like compounds and low levels of acid, followed by the addition of nisin ingredient and potassium sorbate. One example of a bacterial culture that produces a high level of nisin-like compounds and low levels of acid is Lactococcus lactis lactis strain NB-1 (ATCC # ______). Lactococcus lactis lactis strain NB-1 (ATCC # ______) was isolated. This strain was found to be capable of generating high levels of nisin and lower levels of lactic acid as compared to other Lactococcus lactis strains.

Lactococcus lactis is a species of non-sporulating, non-motile, Gram-positive bacteria used extensively in the production of buttermilk and cheese. Lactococcus lactis are cocci that generally group in pairs and short chains, typically about 0.5 to about 1.5 μm in length. When fermenting milk, Lactococcus lactis produces large quantities of lactic acid.

Lactic acid, also known as milk acid, plays a role in several biochemical processes and can be produced via fermentation from lactose. Lactic acid is a carboxylic acid with a chemical formula of C₃H₆O₃. It has a hydroxyl group adjacent to the carboxyl group, making it an alpha hydroxy acid (AHA). In solution, it can lose a proton from the acidic group, producing the lactate ion CH₃CH(OH)COO—. Lactic acid can be used in a variety of food stuffs (such as yogurt) to act as an acidity regulator.

The bacteriocin nisin is produced by fermentation using the bacterium Lactococcus lactis. Commercially it is obtained from natural substrates including milk and is not chemically synthesized. It is used in processed cheese production to extend shelf life by suppressing gram-positive spoilage and pathogenic bacteria. Due to its highly selective spectrum of activity it is also employed as a selective agent in microbiological media for the isolation of gram-negative bacteria, yeast and molds. Two commercially available antimicrobials containing nisin are Nisalpin® and Novasin™ (both from Daniso A/S, Denmark).

Nisin is an inhibitory polycyclic peptide with 34 amino acid residues used as a food preservative. It contains the uncommon amino acids lanthionine, methyllanthionine, dehydroalanine and dehydro-amino-butyric acid. These amino acids are synthesized by posttranslational modifications. In these reactions a ribosomally synthesized 57-mer is converted to the final peptide. The unsaturated amino acids originate from serine and threonine.

Potassium sorbate is a mild preservative used to inhibit molds, and yeasts in many foods, such as cheese, wine, yogurt, and baked goods. It can also be found in the ingredients list of many dried fruit products. In addition, herbal dietary supplement products generally contain potassium sorbate, which acts to prevent mold and microbes and to increase shelf life, and is used in such tiny quantities that there is no known adverse health effects.

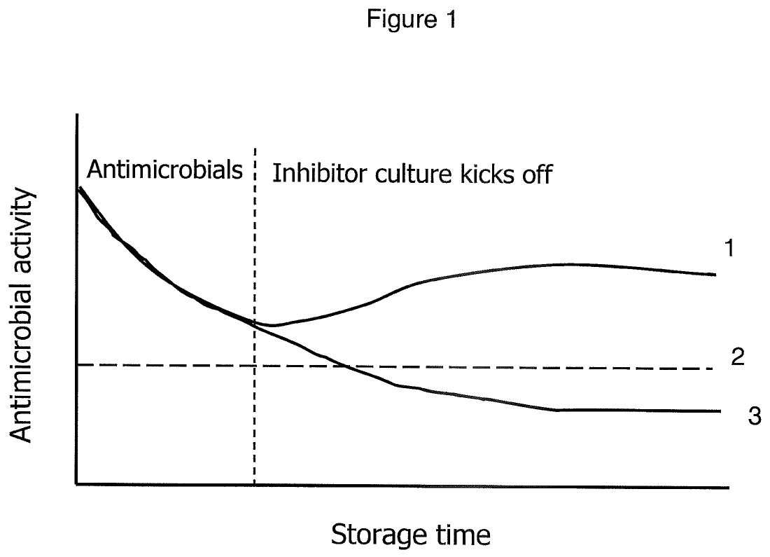

“High level” of nisin-like compounds refers to the generation of nisin-like compounds (and other antimicrobial compounds) at a rate greater than typical or conventional bacterial cultures. FIG. 1 illustrates antimicrobial activity over a period of time for a food product containing antimicrobial ingredients with either the low acid, high nisin producing culture 1 or a conventional culture 3. As shown in FIG. 1, the antimicrobial activity with a conventional culture 3 starts high and is reduced over time until the antimicrobial activity is reduced to a level below an effective level (indicated by line 2 in the figure). The low acid, high nisin producing culture 1, however, starts with a high level of antimicrobial activity, drops slightly (but not below the effective level 2), and then increases until a constant level of antimicrobial activity is reached. This increase is thought to be due to the in-situ and essentially continuous generation of antimicrobials by culture 1 in the product.

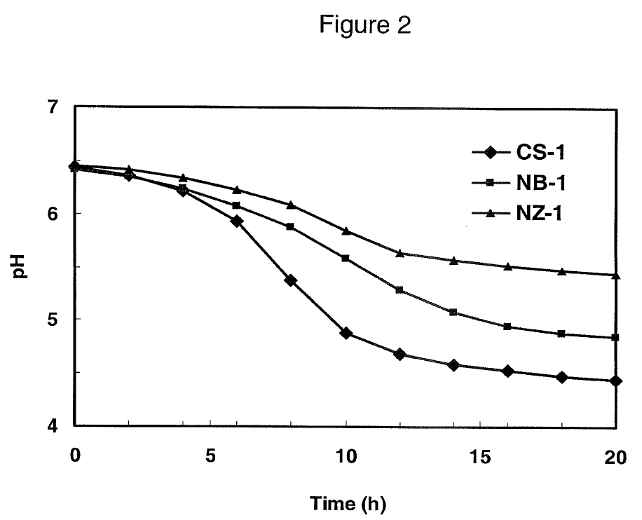

“Low levels” of acid refers to the generation of acid (such as lactic acid) at a rate lower than typical or conventional lactic acid bacterial cultures. FIG. 2 illustrates acid production for three strains of Lactococcus lactis over time. As shown in FIG. 2, strain NB-1 produces a moderate amount of acid in the milk as measured by pH. The pH produced by strain NB-1 at 20 hours at about 30° C. is approximately 4.9. Strain CS-1 produced a pH of approximately 5.5 at 20 hours and strain NZ-1 produced a pH of approximately 4.5 at 20 hours under similar incubation conditions.

Following the ripening process with a low acid, high nisin producing culture, the product may be processed using traditional methods. For example, the product may be set; the curds cut, healed, and cooked; and the curd and whey stirred and drained. Then, potassium sorbate and nisin ingredient are added to the curd. The resulting product can then be packaged and stored at refrigeration temperatures or slightly abused temperatures. For the purposes of describing the invention herein, slightly abused temperatures refers to temperatures in the range of about 45 to about 55° F.

Although not wishing to be limited to theory, it is hypothesized that the high nisin, low acid starting culture cells remain active in continuously generating antimicrobial compounds, but does not aggressively grow in the cheese at refrigerated and slightly abused temperatures. The combined effect of the pre- and in-situ generated antimicrobial metabolites and competitive exclusion from the culture provides effective inhibition against pathogens. It is also hypothesized that the inventive cheese remains microbiologically safe at slightly abused temperatures because the low acid, high nisin producing culture is activated at higher temperatures which causes more antimicrobial compounds to be generated.

Advantageously, although the inventive cheese described herein remains shelf stable for longer and is microbiologically safer, the cheese does not suffer from adverse organoleptic properties. For example, in conventional Queso Fresco cheese, it is desirable to prevent post-process acidification. If post-process acidification occurs, the acid produced during storage will result in rapid acidification of the cheese and the characteristic crumbly texture and melt restriction properties are lost and an undesirable fermented flavor will develop. By using the methods described herein, risk of post-process acidification is mitigated or eliminated by using a starting culture that is a low acid producer. Thus, the methods described herein can be used to produce a Queso Fresco cheese that may be stored for an extended shelf life without losing the typical physical and organoleptic characteristics of Queso Fresco cheese.

A better understanding of the present embodiment and its many advantages may be clarified with the following examples, given by way of illustration.

### EXAMPLE 1

### Preparation of Queso Fresco Cheese

A Queso Fresco cheese with enhanced microbial stability was prepared as described hereinbelow. A lactic acid bacteria producing a low level of acid and a high level of nisin-like antimicrobials was used as starter culture. The culture, in an amount of 2×10⁶ cfu/g, was inoculated into skim milk and incubated at 30° C. for 20 hours. The cultured milk was then used to make Queso Fresco cheese as follows:

1. Add CaCl₂ (0.01% of 45% stock solution, diluted in deionized water) to milk in vat;

2. Slowly heat milk in vat with agitation—target about 88° F. (range of about 86 to about 90° F.);

3. Add the pre-made starter culture at 2%;

4. Hold 15 min with agitation;

5. Add rennet (0.01% of 2× Chymax, diluted in deionized water);

6. Hold 40 minutes with no agitation;

7. Cut curd and heal for 10 minutes—no agitation;

8. Slowly heat curd and whey to about 106° F. over about 20 min—with agitation;

9. Hold for 5 minutes with no heat and no agitation;

10. Pump curd and whey to drain table, stir and drain whey (curd pH 6.2-6.5);

11. Mix in salt (2% of curd), potassium sorbate (0.05%) and Nisaplin (0.05%);

12. Let stand for 5 minutes;

13. Pack cheese into individual packages/casings; and

14. Refrigerate the cheese.

### The resulting cheese had a moisture content of about 48% and a pH of about 6.3.

### EXAMPLE 2

### Microbial Stability of Queso Fresco at Refrigeration Temperature

The microbial stability of the inventive cheese from Example 1 above was compared to a commercial Queso Fresco cheese under refrigeration conditions. Two pathogens, which are capable of growing under refrigeration conditions, were used. The psychrotrophic pathogens included both Gram-positive (Listeria monocytogenes) and Gram-negative bacteria (Escherichia coli). The L. monocytogenes used in this example was a mixture of 6-strains isolated from both dairy and meat outbreaks and food processing environment. The E. coli used in this example was a generic species obtained from American Type Culture Collection with the strain designation of ATCC 51739.

A commercial Queso Fresco cheese made with traditional starter culture and obtained from a local supermarket was used as control in this example. Both the inventive cheese sample and the control sample were inoculated with L. monocytogenes and E. coli and were stored at 45° F. for a period of 10 weeks. During refrigeration storage, samples were taken for enumeration of Listeria monocytogenes on MOX medium (modified Oxford Medium) supplemented with a Listeria-selective additive and of E. coli on a VRB (violet red bile) medium. The results of the growth of L. monocytogenes and E. coli in the cheeses at 45° F. are summarized in Tables 1 and 2.

TABLE 1
Growth of L. monocytogenes in Queso Fresco cheese during storage at 45° F.

| | Time (weeks) | | | | | | | |
|---|---|---|---|---|---|---|---|---|
| Treatment | 0 | 1 | 2 | 3 | 4 | 6 | 8 | 10 |
| Control | 1.5 × 10² | 4.0 × 10² | 7.8 × 10³ | 4.5 × 10³ | 1.0 × 10⁶ | 6.6 × 10⁶ | 2.5 × 10⁵ | 4.0 × 10⁵ |
| Inventive | 1.5 × 10² | 3.0 × 10² | 10 | 3 | 3 | 6 | 44 | 50 |

### Values in the above table are reported in colony forming units (cfu) per gram of cheese.

The data shows that the control sample supported a rapid growth of L. monocytogenes while the inventive sample effectively inhibited the growth of Listeria monocytogenes at refrigeration temperature.

TABLE 2
Growth of E. coli in Queso Fresco cheese during storage at 45° F.

| | Time (weeks) | | | | | | | |
|---|---|---|---|---|---|---|---|---|
| Treatment | 0 | 1 | 2 | 3 | 4 | 6 | 8 | 10 |
| Control | 2.1 × 10² | 3.0 × 10⁷ | 5.0 × 10⁷ | 5.1 × 10⁶ | 6.7 × 10⁶ | 1.0 × 10⁶ | 2.8 × 10⁵ | 1.0 × 10⁴ |
| Inventive | 2.1 × 10² | 35 | 20 | 16 | 5 | 2 | 32 | 24 |

### Values in the above table are reported in colony forming units (cfu) per gram of cheese.

The data shows that the control sample supported a rapid growth of E. coli while the inventive sample effectively inhibited the growth of E. coli at refrigeration temperature.

### EXAMPLE 3

### Microbial Stability of Queso Fresco at Abused Temperature

The microbial stability of the inventive cheese from Example 1 above was compared to a commercial Queso Fresco cheese at a slightly abused temperature (55° F.). The cheese made in Example 1 was inoculated with L. monocytogenes and E. coli and was stored at 55° F. for a period of 8 weeks. A commercial Queso Fresco (the same sample as used in Example 2) made with traditional starter culture was used as a control to serve as a comparison. Samples were taken for L. monocytogenes and E. coli analysis periodically. The following results were obtained (Tables 3 and 4).

TABLE 3
Growth of L. monocytogenes in Queso Fresco cheese during storage at 55° F.

| | Time (weeks) | | | | | | |
|---|---|---|---|---|---|---|---|
| Treatment | 0 | 1 | 2 | 3 | 4 | 6 | 8 |
| Control | 85 | 1.7 × 10⁴ | 2.6 × 10⁷ | 1.3 × 10⁸ | 8.2 × 10⁷ | 9.0 × 10⁷ | 4.5 × 10⁷ |
| Inventive | 85 | 30 | 20 | 40 | 2 | 1 | 5 |

### Values in the above table are reported in colony forming units (cfu) per gram of cheese.

The data shows that the control sample supported a rapid growth of L. monocytogenes while the inventive sample effectively inhibited the growth of Listeria monocytogenes at 55° F.

TABLE 4
Growth of E. coli in Queso Fresco cheese during storage at 55° F.

| | Time (weeks) | | | | | | |
|---|---|---|---|---|---|---|---|
| Treatment | 0 | 1 | 2 | 3 | 4 | 6 | 8 |
| Control | 48 | 54 | 70 | 2.1 × 10² | 1.0 × 10² | 3.1 × 10² | 5.0 × 10² |
| Inventive | 48 | 8 | 1 | 4 | 5 | <1 | <1 |

### Values in the above table are reported in colony forming units (cfu) per gram of cheese.

The data shows that the control sample supported a rapid growth of E. coli while the inventive sample effectively inhibited the growth of E. coli at 55° F.

### EXAMPLE 4

### Stability of pH and Melt Restriction of Queso Fresco During Storage

The inventive cheese made in Example 1 was stored at 45° F. and the pH of the cheese was monitored over time. A commercial Queso Fresco cheese (the same sample as used in Examples 2 and 3) made with traditional starter culture was used as a comparison. The following results were obtained.

TABLE 6
Change in pH of Queso Fresco cheese during storage at 45° F.

| | Time (weeks) | | | | | | | |
|---|---|---|---|---|---|---|---|---|
| Treatment | 0 | 1 | 2 | 3 | 4 | 6 | 8 | 10 |
| Control pH | 6.36 | 6.47 | 6.15 | 6.06 | 6.06 | 6.02 | 5.98 | 5.89 |
| Inventive pH | 6.25 | 6.22 | 6.02 | 5.98 | 5.99 | 6.01 | 5.99 | 6.01 |

The pH data indicates that by using the inventive low acid producing starter culture, the cheese did not have significant acidification during storage under refrigeration conditions.

### EXAMPLE 5

### Meltability of Queso Fresco

The meltability of the cheese prepared in example 1 was tested using the boiling method. Pre-cut cheese plugs were placed on a glass plate and covered with a glass lid. The plate was put on the surface of boiling water to heat for 8 min. The inventive cheese was compared to a commercial Queso Fresco cheese (the same sample as used in Examples 2-4). The melt restriction property of the inventive sample was close to that of the model target.

### EXAMPLE 6

### Sensory Evaluation of the Inventive vs. Traditional Queso Fresco

A formal consumer evaluation was performed among a group of 50 Hispanic consumers screened to be past one month purchasers or users of Queso Fresco who are Mexican origin between 21 and 64 years of age. The products were evaluated in several categories including general rating, purchase intent, flavor, texture, melting and appearance. The assessment results are shown in Table 7.

TABLE 7
Consumer sensory evaluation scores of Queso Fresco cheese

| | | |
|---|---|---|
| | Control | Inventive |
| Category | (no antimicrobials) | cheese |

General ratings:

| | | |
|---|---|---|
| Overall opinion (9 pt.) | 7.04 | 7.22 |
| Expectation (“better or same” %) | 80 | 86 |
| Purchase intent (5 pt.) | 3.96 | 3.96 |

Flavor:

| | | |
|---|---|---|
| Overall flavor liking (9 pt.) | 6.94 | 7.12 |
| Flavor strength (“just right” %) | 64 | 68 |
| Milky/dairy (“just right” %) | 66 | 70 |
| Saltiness (“just right” %) | 78 | 60 |
| Sourness (“just right” %) | 74 | 62 |
| Moistness (“just right” %) | 62 | 70 |
| After taste (“moderate” %) | 60 | 52 |
| Pleasantness after taste (%) | 87 | 84 |

Texture/Melting:

| | | |
|---|---|---|
| Overall texture liking (9 pt.) | 7.02 | 7.18 |
| Crumbliness liking (9 pt.) | 7.18 | 7.52 |
| Creaminess (“just right” %) | 34 | 42 |
| Firmness (“just right” %) | 60 | 54 |
| Smoothness (“just right” %) | 60 | 62 |
| Melting liking (9 pt.) | 6.60 | 6.68 |

Appearance:

| | | |
|---|---|---|
| Overall appearance liking (9 pt.) | 7.08 | 7.20 |
| Color (“just right” %) | 82 | 74 |
| Moist appearance (“just right” %) | 62 | 68 |

The consumer evaluation results suggest that the inventive sample was at parity to the control (traditionally made base cheese without any inhibitors). The inventive cheese did not significantly change the overall quality profiles of the cheese, nor impact consumer acceptance of the cheese.

All references (including publications, patents, patent publications, and patent applications) cited herein are incorporated by reference. All percentages throughout this specification are by weight percent unless otherwise specified.

What is claimed is:

1. A fresh cheese product comprising:
milk ripened with a low acid, high antimicrobial producing starter culture;
potassium sorbate and nisin ingredient; and
a pH in the range of about 5.8 and about 6.8, wherein the fresh cheese is stable for at least 30 days and exhibits organoleptic properties similar to traditional fresh cheeses.

2. The fresh cheese product of claim 1 wherein the starting culture is Lactococcus lactis.

3. The fresh cheese product of claim 2 wherein the bacterial strain is assigned ATCC ______.

4. The fresh cheese product of claim 1 wherein potassium sorbate is added in the range of about 0.01 to about 0.1 percent.

5. The fresh cheese product of claim 1 wherein nisin ingredient is added in the range of about 0.01 to about 0.1 percent.

6. The fresh cheese product of claim 5 wherein the nisin ingredient is Nisaplin.

7. A method of making a fresh cheese product comprising:
adding to milk a low acid, high antimicrobial producing starter culture; and
adding to the ripened milk potassium sorbate and nisin, wherein the fresh cheese has a pH in the range of about 5.8 and about 6.8 and is stable for at least 30 days and exhibits organoleptic properties similar to traditional fresh cheeses.

8. The fresh cheese product of claim 1 wherein the starting culture is Lactococcus lactis.

9. The fresh cheese product of claim 8 wherein the bacterial strain is assigned ATCC ______.

10. The fresh cheese product of claim 7 wherein potassium sorbate is added in the range of about 0.01 to about 0.1 percent.

11. The fresh cheese product of claim 7 wherein nisin ingredient is added in the range of 0.01 to 0.1 percent.

12. The fresh cheese product of claim 11 wherein the nisin ingredient is Nisaplin.

13. A biologically pure culture of Lactococcus lactis having all of the characteristics of strain NB-1, ATCC ______.

14. A biologically pure culture of low acid, high antimicrobial producing bacteria.

15. The biologically pure culture of claim 14 wherein the bacteria is Lactococcus lactis.
